(19) United States
(12) Patent Application Publication
(10) Pub. No.: US 2013/0079862 A1
(43) Pub. Date: Mar. 28, 2013

(54) ELECTRODE ARRANGEMENT

(71) Applicant: CERBOMED GMBH, Erlangen (DE)

(72) Inventor: Jens ELLRICH, LANGENSENDELBACH (DE)

(73) Assignee: CERBOMED GMBH, Erlangen (DE)

(21) Appl. No.: 13/683,727

(22) Filed: Nov. 21, 2012

Related U.S. Application Data
(63) Continuation of application No. PCT/EP2011/002453, filed on May 18, 2011.

(30) Foreign Application Priority Data
May 28, 2010 (DE) 10 2010 021 877.4
Nov. 4, 2010 (DE) 20 2010 014 951.7

(51) Int. Cl. A61N 1/05; A61N 1/04

(52) U.S. Cl. CPC A61N 1/0541; A61N 1/0456
USPC 607/137; 607/56

(57) ABSTRACT

The invention relates to an electrode arrangement to be attached on and/or in the ear of a human, wherein the electrode arrangement is designed to exert a transcutaneous electric stimulation stimulus onto the surface of the ear and wherein the electrode arrangement has at least one stimulation electrode and at least one reference electrode. In order to achieve an improved transcutaneous stimulation, especially of the Vagus nerve, the invention provides that the at least one stimulation electrode contacts the surface of the ear via a first contact surface and that the at least one reference electrode contacts the surface of the ear via a second contact surface, wherein the second contact surface is at least three time the size of the first contact surface.

This application is a continuation of PCT/EP2011/002453 filed May 18, 2011, which claimed the priority of German Patent Applications DE 10 2010 021 877.4 filed May 28, 2010 and DE 20 2010 014 951.7 filed Nov. 4, 2010, all three applications are incorporated herein by reference.

The invention relates to an electrode arrangement to be attached on and/or in the ear of a human, wherein the electrode arrangement is designed to exert a transcutaneous electric stimulation stimulus onto the surface of the ear, wherein the electrode arrangement has at least one stimulation electrode and at least one reference electrode, wherein the at least one stimulation electrode has an arcuated structure and contacts the surface of the ear via a first contact surface and that the at least one reference electrode has an oval or drused structure and contacts the surface of the ear via a second contact surface, wherein the second contact surface is at least three times the size of the first contact surface.

It is generally known to take influence on the neurophysiological and neuroelectrical quality through invasive and non-invasive stimulation of the nerves and thereby on the function of the simulated nerves. Hereby different conditions of sickness can be treated. Numerous devices exist both for the invasive and the non-invasive stimulation.

The present invention is basing upon the method of the transcutaneous electrical stimulation of the nerves. At this method pulse currents of different current forms, amplitudes, pulse durations and frequencies are administered through the skin on different nerves and change their status parameter in an advantageous way.

An electrode arrangement of the kind mentioned in the beginning is known from DE 10 2005 003 735 B4. Here a device is described for the transcutaneous stimulation of the Vagus nerve of the human body which has a bow-shaped extension for the insertion into the ear canal, which has an electrode head on its end that is to be inserted into the ear canal. Here, two point-shaped electrodes are arranged in distance along the direction of the axis of the ear canal. With this pre-known solution an effective transcutaneous stimulation can already occur particularly in the area of the ear canal where the Vagus nerve runs. However the areas to be stimulated are limited.

A generic solution is shown also in U.S. Pat. No. 4,267,838. Similar and other stimulation devices are described in WO 92/08516, in U.S. Pat. No. 4,966,164 and in US 2006/0064139 A1.

Another electrode arrangement is disclosed in DE 10 2006 023 824 A1 which can be placed within the Pinna of the ear. Electrodes are provided here at the end of elastically developed holding elements, by which the electrode arrangements can be clamped firmly within the ear.

From US 2003/0195588 A1 an electrode head is known according to a certain kind of ear canal plug, which has electrodes in the form of closed rings. Also herewith a transcutaneous stimulation is possible. However, based on the relatively steep structure of the electrode head, there are reductions when it comes to the flexibility of the electrodes of the inner surface of the ear canal.

A basically other nerve stimulation is described in U.S. Pat. No. 3,449,768 and in U.S. Pat. No. 5,649,970. Stimulation electrodes are used here which are implanted within the area of the ear of the patient.

At the pre-known solutions the electrodes, that is the stimulation electrode and the reference electrode, have a substantial equal form and size, as far as the here interesting technology of the transcutaneous nerve stimulation in classification to the stimulation with implanted electrodes is affected. Thereby, for example two metallic electrodes with ball-shaped surfaces are used, which are arranged in a defined distance. It is also known, that ring-shaped metallic elements are used as electrodes, which are arranged also in a defined distance to each other.

It turned out that this embodiment of the electrodes doesn't always bring forth an optimal treatment result. In fact, another conception of the electrode form and size seems to bring forth a better stimulation result. In this connection it illustrates a particular attention and problem respectively, that it is not without problems to reach reproducibly a defined intensity of the transcutaneous nerve stimulation, as a result of the hairiness of the skin surface and as a result of a grease film on the skin surface, especially existing in the area of the ear.

Thus, it is an object of the invention to develop an electrode arrangement of the generic kind in such a way to stay abreast of the mentioned disadvantage. Accordingly, an electrode arrangement shall be created which is designed in such a way that an improved treatment result can be reached at the application of a transcutaneous electrical stimulation impulse. Thereby, it is especially aimed to obtain an anergy of the stimulation as high as possible with respect to the hairiness of the skin surface and where appropriated a grease film on the same.

The solution of this object by the invention is characterized in that at least one of the electrodes contacts the surface of the ear via a contact surface, wherein the electrode is so designed that it covers at least 50% of the surface of the Cymba conchae of the ear.

Preferably, the second contact surface is at least five times the size of the first contact surface.

Thereby, the at least one stimulation electrode acts preferably as a cathode and the at least one reference electrode as an anode in the electrical circuit which is closed during the transcutaneous stimulation. As is well-known the cathode is that electrode, where reduction reactions take place and electrons are emitted. The cathode can have a negative polarity as in the case of an electrical consumer or a positive polarity as in the case of an electrical generator, e.g. a voltage source. The cathode it the counter electrode to the anode. Cations travel to the cathode and anions to the anode.

The stimulation electrode can be adapted with its arcuated structure to the outline form of the Tragus of the ear. This can be the outer side of the Tragus as well as its inner side. The stimulation electrode has thereby preferably a falx-shaped structure.

The reference electrode can be adapted with its oval or drused structure to the form of a substantial flat area of the surface of the Pinna of the ear.

The at least one stimulation electrode and the at least one reference electrode are preferably arranged with a distance between 5 mm and 50 mm to another during intended use.

The stimulation electrode and the reference electrode can consist of at least one metallic body. The metallic body can be arranged at or in a carrier body which consists of an elastic material. Thereby, the elastic material is preferably a synthetic material, especially a bio compatible elastomere material, particularly preferred silicone or a material, which comprises silicone.

The electrode arrangement can consist at least partially of a conductive synthetic material. It can also consist of a synthetic material which is provided at least partially with a conductive surface. The conductivity of the synthetic material and of the synthetic material surface respectively can be used for realization of the electrodes.

Thereby, the electrode or the electrode carrier is preferably so designed that it covers at least 80% of the surface of the Cymba conchae of the ear.

Furthermore, it can be provided that a further electrode or an electrode carrier which bears the same contacts the surface of the ear via a further contact surface, wherein the electrode or the electrode carrier is so designed that it covers a part of the Antihelix of the ear.

Thus, the invention provides different large and preferably also different sized and different poled electrodes. Thereby, it is preferably provided that the stimulation electrode which acts as a cathode is placed directly on the position of the biggest subcutane concentration of the auricular Vagus nerve so that nerves which lay underneath are depolarized by the delivered negative charge preponderance. A respectively higher current density results from the small electrode surfaces compared with the reference electrodes, whereby the probability rises that the Vagus branches are depolarized, which are mentioned above. The reference electrode which acts as an anode will be then arranged in distance to the stimulation electrode on the nearby skin. In doing so a too large distance shall be avoided, so that not a lot of unnecessary body tissues will be flown through by the current during the transcutaneous stimulation and not a too high current will be needed.

Furthermore it is advantageous that due to the suggested design of the electrode arrangement a very easy application is possible by the user, because preferably there is no insertion necessary of a section of the electrode arrangement into the ear canal.

By, the suggested choice of the surface relations between the electrodes it will be particularly achieved in an advantageous way that a relatively high anergy of the transcutaneous nerve stimulation is given against the hairiness and the grease film of the skin surface.

In the drawings embodiments of the invention are depicted. It shows:

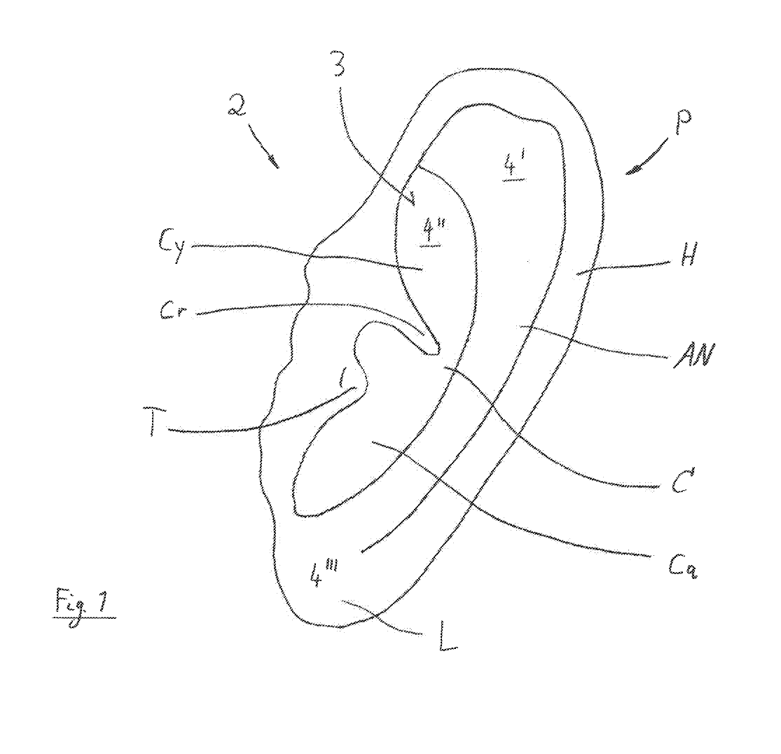

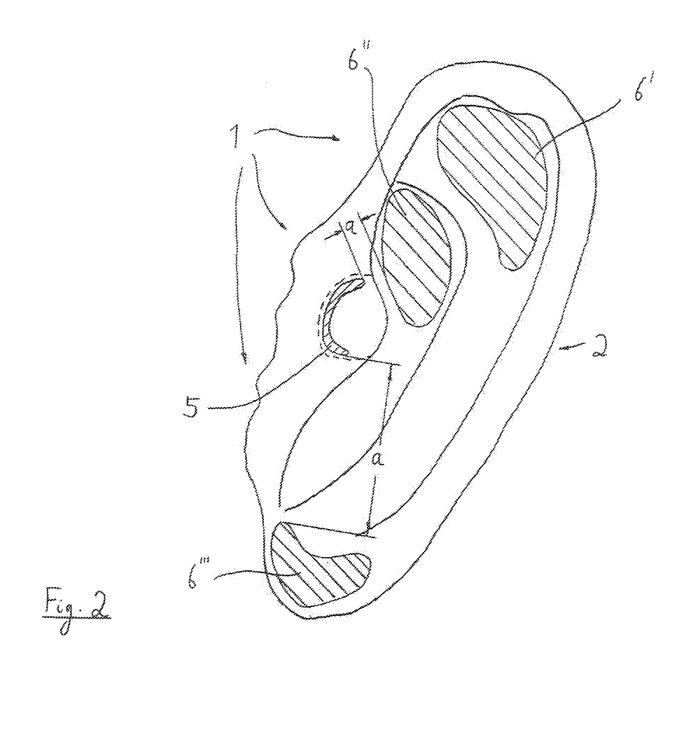

FIG. 1 a concha (Pinna of a human,

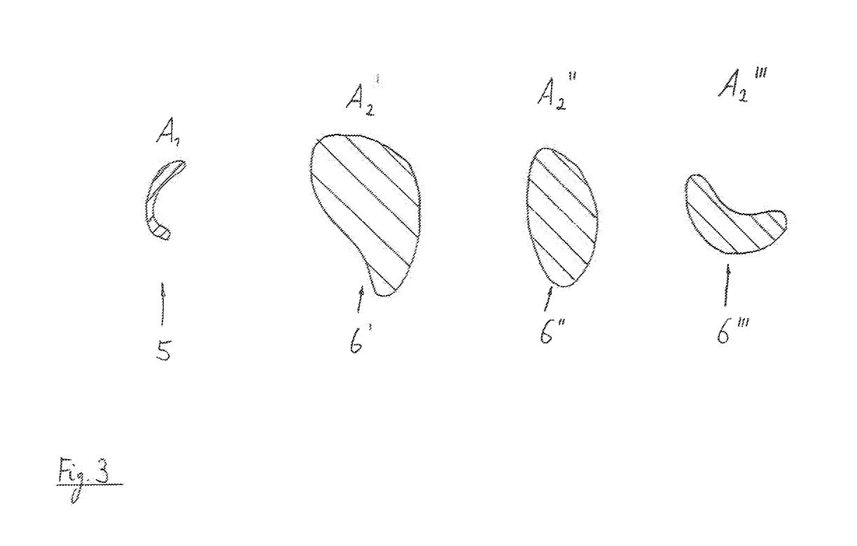

FIG. 2 the concha with an electrode arrangement which is put upon a defined area of the ear to carry out a transcutaneous stimulation,

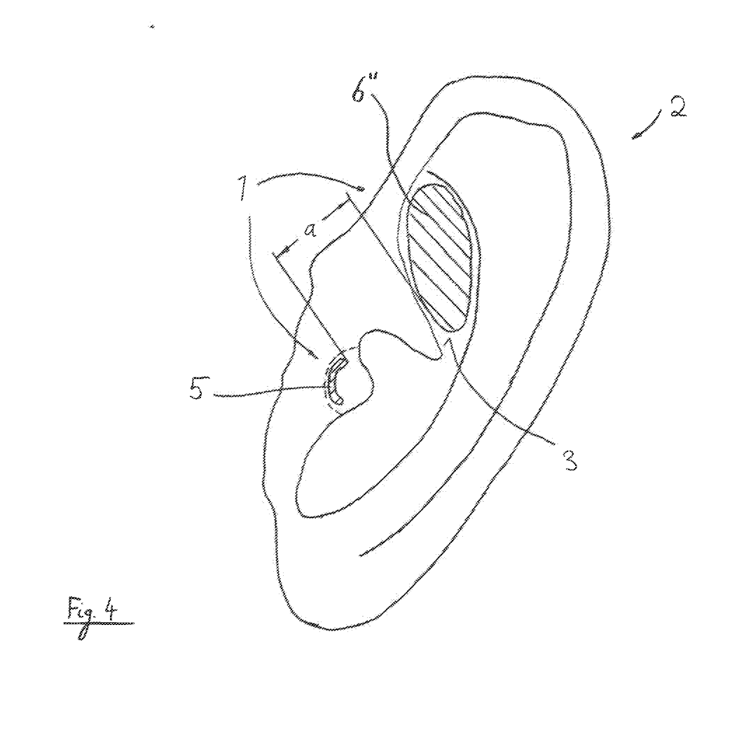

FIG. 3 the electrodes which are applied at the electrode arrangement according to FIG. 2,

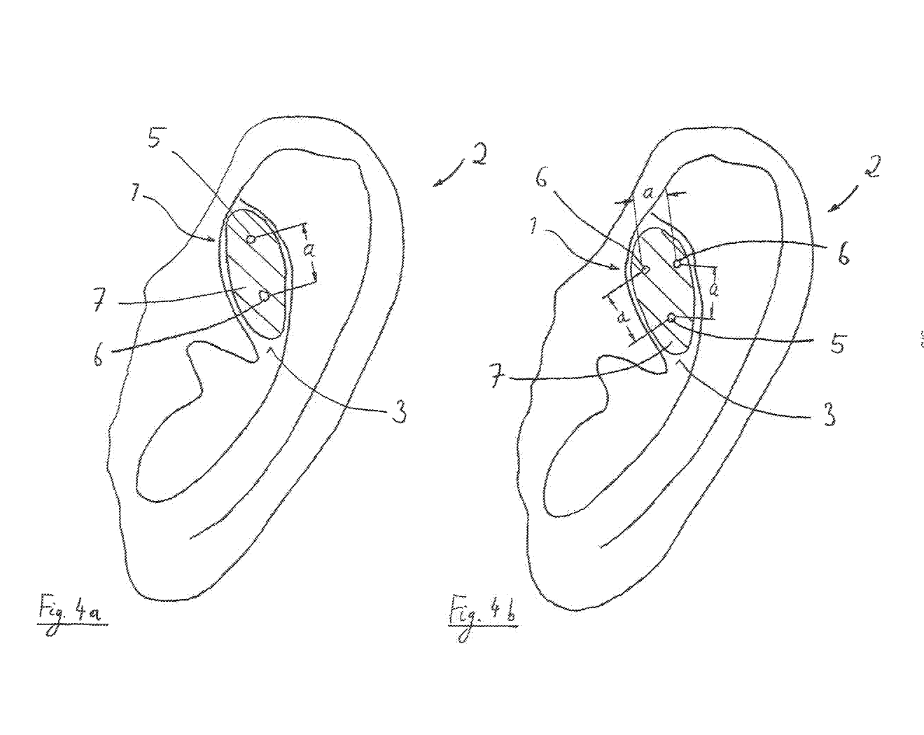

FIG. 4 the concha with an electrode arrangement which especially is put upon the region of the Cymba concha to carry out a transcutaneous stimulation,

FIG. 4a an embodiment of the electrode arrangement which is alternative to FIG. 4 and

FIG. 4b a further embodiment of the electrode arrangement which is alternative to FIG. 4.

In FIG. 1 an (outer) ear 2 of a human is shown, which form is defined by the Pinna (ear choncha) P. The Pinna P contains the Helix H and Antihelix AN as known; the Concha C is arranged centrally which is confined sideways from the Tragus T. In the lower area there is the Lobule L. The Concha C divides itself in an upper and lower part; both parts are separated to each other from the Crus helicis Cr. The upper part of the Concha C is the Cymba conchae Cy, the lower part is the Cavum conchae Ca.

Within the conditions of the present invention it is provided that particular areas of the ear 2 are exposed to a transcutaneous stimulation. For the installation of a stimulation electrode, which operates as a cathode, a surface 3 of the ear 2 is provided, wherein it is here the inner side of the Tragus T. For the arrangement of a reference electrode which operates as an anode, several zones can be provided alternatively or additively, wherein a surface 4′ in the upper part of the Antihelix AN, a surface 4″ in the upper part of the Concha C and/or a surface 4′″ the region of the Lobule L is preferred.

In FIG. 2 it is shows how an electrode arrangement 1 on and within the ear 2 respectively is placed to perform a transcutaneous stimulation upon the surfaces 3, 4.

Here the electrode arrangement 1 is only depicted regarding to their electrodes 5 and 6. Further items (possibly housing and electrical connections) are not designed. The necessary means are well-known in the state of the art so that they don't have to be described further here. Exemplary it will be pointed to DE 10 2005 003 753 B4 of the applicant and reference is made explicitly hereunto.

Using the electrodes 5, 6 a transcutaneous electrical nerve stimulation can be carried out upon the surfaces 3 and 4 of the ear (s. FIG. 1) and especially there where the Vagus nerve runs. Between the stimulation electrode 5 and the (at least one) reference electrode 6 an electrical potential will be created for this purpose.

As it can be recognized in FIG. 2 and FIG. 3 the stimulation electrode 5 has a bended, lunated shape in the embodiment. The surface of the stimulation electrode 5 is marked with A₁, with which it contacts the surface 3 of the ear 2, in the present case the inner side of the Tragus T.

The lunated shape of the stimulation electrode 5 is of course not compulsive. In principle, an asymmetrical electrode geometry can be used within the whole area of the outer ear (thus also Cymba, ear canal, Tragus, etc.).

The reference electrodes 6 have a shape which is adjusted to the area and surface of the ear 2 respectively, where they shall be placed. Oval structures (as in example of the electrode 6″) or drused structures (as in example of the electrode 6′″) can be provided.

The three demonstrated reference electrodes 6′, 6″ and 6′ can be used alternatively or additively. Each of the electrodes 6′, 6″, 6′″ contact the surface 4′, 4″ and 4″ of the ear 2 respectively with a contact surface which is identified with A₂ (respectively A₂′, A₂″ and A₂′″ in FIG. 3).

It is essential that the second contact surface A₂ is remarkably larger than the first contact surface A₁. This means precisely that the surface A₂ is at least 3 times as big as the surface A₁. As it can be seen on the basis of the illustration according to FIG. 3, a significant larger relation of the surfaces is provided, in the embodiment a relation of at least 1:5.

The electrodes 5 and 6 are arranged in a distance a within the ear 2. The minimal distance is in most cases 5 mm. Distances up to 50 mm can be also provided.

In any case it will be pursued that the stimulation electrode 5 is arranged directly to the place of the largest subcutaneous concentration of the auricular Vagus nerve. The reference electrode(s) 6 will be placed according to the distance a to the stimulation electrode 5 in the nearby area. The distance a will be chosen like that, that not a lot of unnecessary body tissues will be flown through by the current, on the other hand also not a too high current rating will be needed.

The electrodes 5, 6 which consist of metal can be embedded into an elastomer material, wherefore a soft plastic material suits (for example silicone or polyurethane), wherein a shore-grade within the area between 30 and 50 can be provided.

The use of electrical conducting plastic material is also possible instead of metallic electrodes which makes the electrodes smoother and more adjustable.

The electrodes 5, 6 can be integrated in a holding arrangement which is not shown, which will be inserted into the ear, whereby all provided electrodes 5, 6 get into their intended position.

For the arrangement of the reference electrode which functions as an anode, the Cymba conchae Cy is provided according to the embodiment of FIG. 4, which will be covered from the reference electrode 6″ to at least 50% of its surface 4″. The coverage of the Cymba conchae Cy is preferentially even bigger, particularly more than 80%. It turned out that an electrode 6″ which is applied here, has an optimal stimulation effect.

A further embodiment of the suggested electrode arrangement is illustrated in FIG. 4a and FIG. 4b. It is essential here that the electrode arrangement covers and contacts respectively exclusively the area of the Cymba conchae Cy.

The part of the stimulation device which is provided with electrodes has here an electrode carrier 7 which carries the electrodes 5, 6 that are needed for the stimulation.

The two FIGS. 4a and 4h show two possibilities for the arrangement of the electrodes 5, 6 upon the electrode carrier Only point-shaped metallic electrodes are depicted schematically, which provide a distance between themselves and therefore stimulate the area of the Cymba conchae Cy which lies between them, after the admission with an electrical power.

Of course diverse variations are possible relating to the amount and the arrangement of the electrodes 5, 6 upon the electrode carrier 7.

Thus, it is essential at the solution according to the FIGS. 4a and 4b, that simply the area of the Cymba conchae Cy is contacted from the stimulation electrodes. An alternative solution, which is not illustrated, places itself on it, to stimulate exclusively the area of the Antihelix accordingly in an analog way. A further alternative embodiment provides that the area of the Cymba conchae and the Antihelix will be stimulated combined with an electrode carrier 7.

Accordingly the demonstrated solutions according to FIG. 4a and FIG. 4b place themselves on it that the electrodes 5, 6 contact the surface 3 of the ear 2 over a contact surface, wherein the electrodes 5, 6 are developed in such a way that they or an electrode carrier 7 which carries them cover at least 50% of the surface of the Cymba conchae of the ear 2 and wherein no other further area of the ear 2 is provided with an electrode.

The analogue situation to the alternative described solution applies with regard to the stimulation of the Antihelix.

### LIST OF REFERENCES

1 Electrode Arrangement
2 Ear
3 Surface of the ear
4; 4′
4″, 4′″ Surface of the ear
5 Stimulation electrode
6 Reference electrode
6′ Reference electrode
6″ Reference electrode
6′″ Reference electrode
7 Electrode carrier
A₁ First contact surface
A₂ Second contact surface
A₂′ Second contact surface
A₂″ Second contact surface
A₂′″ Second contact surface
a Distance
AN Antihelix.
C Concha
Ca Cavum conchae
Cy Cymba conchae
Cr Crus helicis
H Helix
L Lobule
P Pinna
T Tragus

1. An electrode arrangement to be attached on and/or in the ear of a human, wherein the electrode arrangement is designed to exert a transcutaneous electric stimulation stimulus onto the surface of the ear, the electrode arrangement comprising:
at least one stimulation electrode and at least one reference electrode,
wherein the at least one stimulation electrode has an arcuated structure and contacts the surface of the ear via a first contact surface,
wherein the at least one reference electrode has an oval or drused structure and contacts the surface of the ear via a second contact surface,
wherein the second contact surface is at least three times the size of the first contact surface,
wherein at least one of the electrodes contacts the surface of the ear via a contact surface, wherein the electrode is so designed that it covers at least 50% of the surface of the Cymba conchae of the ear.

2. The electrode arrangement of claim 1, wherein the second contact surface is at least five times the size of the first contact surface.

3. The electrode arrangement of claim 1, wherein the at least one stimulation electrode acts as a cathode and the at least one reference electrode as an anode in the electrical circuit which is closed during the transcutaneous stimulation.

4. The electrode arrangement of claim 1, wherein the arcuated structure of the stimulation electrode is adapted to the outline form of the Tragus of the ear.

5. The electrode arrangement of claim 4, wherein the stimulation electrode has a falx-shaped structure.

6. The electrode arrangement of claim 1, wherein the oval or drused structure of the reference electrode is adapted to the form of a substantial flat area of the surface of the Pinna of the ear.

7. The electrode arrangement of claim 1, wherein the at least one stimulation electrode and the at least one reference electrode are arranged with a distance between 5 mm and 50 mm to another during intended use.

8. The electrode arrangement of claim 1, wherein the stimulation electrode and the reference electrode consist of at least one metallic body.

9. The electrode arrangement of claim 8, wherein the metallic body is arranged at or in a carrier body which consists of an elastic material.

10. The electrode arrangement of claim 9, wherein the elastic material is a synthetic material, especially a bio compatible elastomere material, particularly preferred silicone or a material, which comprises silicone.

11. The electrode arrangement of claim 1, wherein the electrode arrangement is at least partially of a conductive synthetic material.

12. The electrode arrangement of claim 1, wherein the electrode arrangement is a synthetic material which is provided at least partially with a conductive surface.

13. The electrode arrangement of claim 1, wherein the electrode is so designed that it covers at least 80% of the surface of the Cymba conchae of the ear.

14. The electrode arrangement of claim 1, wherein a further electrode or an electrode carrier which bears the same contacts the surface of the ear via a further contact surface, wherein the electrode or the electrode carrier is so designed that it covers a part of the Antihelix of the ear.
